(19) United States
(12) Patent Application Publication
(10) Pub. No.: US 2012/0277783 A1
(43) Pub. Date: Nov. 1, 2012

(54) Reinforced Dilatation Balloon and Methods

(75) Inventors: Michael Cummins, Galway (IE); Gerard Hession, Castlerea (IE)

(73) Assignee: Medtronic Vascular, Inc., Santa Rosa, CA (US)

(21) Appl. No.: 13/097,006

(22) Filed: Apr. 28, 2011

(51) Int. Cl. A61M 29/02; B29C 43/18

(52) U.S. Cl. 606/191; 156/245

(57) ABSTRACT

A reinforced dilatation balloon, and a method of making, wherein the balloon includes a balloon body having a continuous polymer tube (e.g., generally cylindrical) having a proximal end, a distal end, and an external surface, and a tubular fiber reinforcing sleeve thermally bonded along the length of the sleeve to at least a portion of the external surface of the balloon body.

### BACKGROUND OF THE DISCLOSURE

Surgical procedures employing balloons and medical devices incorporating those balloons (i.e., balloon catheters) are becoming more common and routine. These procedures, such as angioplasty procedures, are conducted when it becomes necessary to expand or open narrow or obstructed openings in blood vessels and other passageways in the body to increase the flow through the obstructed areas. For example, in an angioplasty procedure, a dilatation balloon catheter is used to enlarge or open an occluded blood vessel which is partially restricted or obstructed due to the existence of a hardened stenosis or buildup within the vessel. This procedure requires that a balloon catheter be inserted into the patient's body and positioned within the vessel so that the balloon, when inflated, will dilate the site of the obstruction or stenosis so that the obstruction or stenosis is minimized, thereby resulting in increased blood flow through the vessel.

Many times, once the balloon has been arranged at the vessel narrowing, it is repeatedly inflated and deflated. The inflation, with successive deflation, of the balloon within the vessel (e.g., artery) reduce the extent of the vessel luminal narrowing, and restore a suitable blood flow in the cardiac area, suffering from the stenosis. In some cases, the balloon serves to deliver a stent.

Balloons are typically formed using a thermoplastic or thermoplastic elastomer but the pressure capability and compliance control is limited by the technology, materials, and extrusion design. Reinforced balloons, particularly fiber-reinforced balloons, can provide one or more of the following: greater pressure capability; greater compliance control; and/or greater wall strength upon deployment of a stent or repeated inflation/deflation.

### SUMMARY

The present disclosure provides reinforced dilatation balloons and methods of making In one embodiment, a reinforced dilatation balloon: includes a balloon body having a continuous polymer tube (e.g., generally cylindrical) having a proximal end, a distal end, and an external surface; and a tubular fiber reinforcing sleeve thermally bonded along the length of the sleeve to at least a portion of the external surface of the balloon body. A tubular fiber reinforcing sleeve can cover the entire underlying balloon body. Alternatively, one or more tubular fiber reinforcing sleeves in the form of bands or rings can be disposed on the balloon body in various locations along its length. Tubular fiber reinforcing sleeves are typically, and preferably, made of braided fabric.

In one embodiment, the present disclosure provides a method of making a reinforced dilatation balloon. In one embodiment, the includes: providing a tubular balloon parison comprising a polymeric material; providing a tubular fiber reinforcing sleeve; axially stretching the tubular fiber reinforcing sleeve; applying the tubular, axially stretched, fiber reinforcing sleeve to the tubular balloon parison; applying a matrix polymer to the tubular fiber reinforcing sleeve, the tubular balloon parison, or both, to form a balloon parison/sleeve/matrix polymer construction; providing a mold for forming a balloon; placing the balloon parison/sleeve/matrix polymer construction in the mold; and expanding the balloon parison/sleeve/matrix polymer construction and applying heat to form a fiber reinforced balloon.

As used herein, a “tubular fiber reinforcing sleeve” is a fabric having a continuous annular wall with a passage down the middle. Preferably, the tubular fiber reinforcing sleeve is seamless.

As used herein, “fabric” refers to a pliable material of natural or synthetic fibers. Fibers are used herein in the general sense to include not only materials in the form of fibers, but also materials in the form of filaments, threads, ribbons, wires, and yarns.

Braided fabric refers to a system of fiber architecture in which three or more fibers are intertwined in such a way that no two are twisted exclusively around one another, thereby mechanically interlocking them. Braided fabrics are or several types, including, for example biaxial and triaxial braids. A Biaxial braid consists of two sets of fibers oriented at a fixed angle from the braid axis forming a symmetrical array. A triaxial braid consists of a biaxial braid with added axials in which the fibers are locked together without possible geometric arrangement.

Herein, the terms “distal” and “proximal” are used with respect to a position or direction relative to the treating clinician. “Distal” and “distally” are a position distant from or in a direction away from the clinician. “Proximal” or “proximally” are a position near or in a direction toward the clinician.

The terms “comprises” and variations thereof do not have a limiting meaning where these terms appear in the description and claims.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “a,” “an,” “the,” “at least one,” and “one or more” are used interchangeably.

As used herein, the term “or” is generally employed in its sense including “and/or” unless the content clearly dictates otherwise.

The term “and/or” means one or all of the listed elements or a combination of any of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

The recitations of numerical ranges by endpoints include all numbers subsumed within that range as well as the endpoints (e.g., 1 to 5 includes 1, 1.5, 2, 2.75, 3, 3.80, 4, 5, etc.).

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 shows the balloon catheter in an inflated state with a balloon showing a thermally bonded braided sleeve.

### DETAILED DESCRIPTION OF ILLUSTRATIVE EMBODIMENTS

The present disclosure provides reinforced dilatation balloons, methods of making, and methods of using. In one embodiment, a reinforced dilatation balloon includes: a balloon body having a continuous polymer tube (e.g., generally cylindrical) having a proximal end, a distal end, and an external surface; and a tubular fiber reinforcing sleeve thermally bonded along the length of the sleeve to at least a portion of the external surface of the balloon body. In this context, “thermally bonded” occurs during the balloon forming process (e.g., a blow molding process).

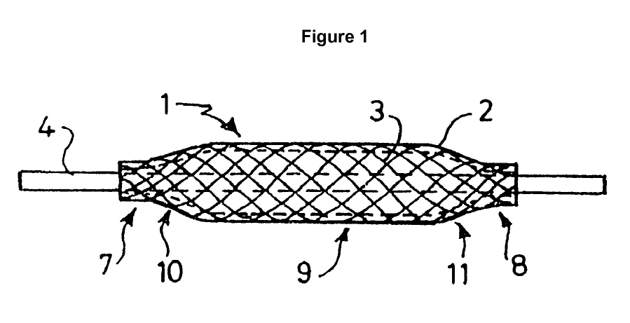

As shown in FIG. 1, an exemplary embodiment of a balloon catheter of the present disclosure includes a reinforced dilatation balloon 1 having a balloon body 2 formed, for example, of a polymeric material. The balloon body 2 includes a continuous polymer tube 3 on a tubular catheter 4, with proximal and distal ends 7 and 8, and having a tubular fiber reinforcing sleeve 9 (e.g., of braided fibers, half of which trace out right-handed parallel helixes, and the other half of which trace out left handed parallel helixes). In this embodiment, the tubular fiber reinforcing sleeve 9 is of a length that substantially matches the length of the balloon body, including the taper/cone regions 10 and 11. The proximal end of the tubular catheter 4 is connected to a source of gas or liquid under pressure which is used to expand the balloon body 2 radially when it has been located at the correct position within a blood vessel.

As shown in exemplary FIG. 1, tubular fiber reinforcing sleeve can cover the entire length of the underlying balloon body. Alternatively, one or more tubular fiber reinforcing sleeves (preferably, a plurality of tubular fiber reinforcing sleeves) in the form of bands or rings can be disposed on the balloon body. When a plurality of reinforcing sleeves is used, they can be disposed in various locations along the length of the balloon body. In certain preferred embodiments, such reinforcing sleeves are uniformly distributed along the length of the balloon body.

The tubular fiber reinforcing sleeve can be made of braided fabric. In certain embodiments, a typical tubular fiber reinforcing sleeve has a very high porosity, i.e., a relatively large amount of open space (e.g., between fibers), although a wide range of porosities is possible. Such porosity can have variously shaped open spaces. In certain embodiments, a tubular fiber reinforcing sleeve can have adjacent fibers with essentially no porosity.

Preferably, the tubular fiber reinforcing sleeve is made of a fabric that has a sufficient stretchiness so that it can be stretched longitudinally (with reduction in OD) prior to insertion into a mold and then it can be stretched radially (with reduction in length) to a point (coincident with the mold ID) whereupon the fibers of the fabric ‘lock out’ and prevent further radial expansion. This can be accomplished, for example, using a braided (helical braid type) fabric where the fibers reach the ‘critical braid angle’ of 54 degrees at the fabric tube diameter at which it is to lock out. Thus, a tube of fabric of braid angle 54 degrees is preferably stretched longitudinally (with decreasing braid angle) and then caused to grow radially in the balloon mold until the critical angle is once again reached. The resultant balloon will thus resist further radial growth under pressurization (outside the mold).

Due to the thermal bonding between the tubular fiber reinforcing sleeve and the external surface of the balloon body, the fiber reinforcement is integral with the material of the wall of the balloon so that it moves with the wall as the balloon body is inflated. The fiber reinforcing sleeve is substantially fixed with respect to the wall material and does not slide or move significantly with respect to the wall material. Such a fixed or integral reinforcement provides enhanced support for the wall material and provides improved restraint to radial over-expansion of the wall material, as compared to braiding or other reinforcement which is free to move or be rearranged relative to the flexible wall material in response to inflation of the balloon.

A sleeve (whether it is one tubular fiber reinforcing sleeve covering the entire length of the underlying balloon body, or a plurality of tubular fiber reinforcing sleeves in the form of bands or rings disposed on the balloon body) is thermally bonded along the length of the sleeve to at least a portion of the external surface of the balloon body, as opposed to having just the ends of the sleeve bonded to the balloon. Having a sleeve thermally bonded along its entire length is advantageous at least because it is believed that this aids in balloon wrapping (only one layer to be wrapped versus two independent layers) and this gives lower overall profile since it would eliminate free space between layers.

In certain embodiments of the disclosure, the fibers of the tubular fiber reinforcing sleeve extend around the longitudinal axis of the balloon body in crossed helical strands to form a braid. The braiding could, however, have other crossed or woven configurations. Preferably, the fiber arrangement allows the braid to stretch longitudinally. This can be accomplished, for example, using helical windings of very specific angle (e.g., 54 degrees).

The desired form and nature of material used to form the fibers of the tubular fiber reinforcing sleeve can readily be determined by simple trial and error tests having regard to the desired geometry of the rest and deployed states of the balloon and the balloon radial expansion forces which the tubular fiber reinforcing sleeve is to resist.

The fibers of the tubular fiber reinforcing sleeve can be made from an inelastic material. However, the reinforcement configuration (e.g., braided or knitted fabrics) may be of a form that allows for limited radial stretching. Thus, the reinforcement constructed from the inelastic fibers need not itself be inelastic.

The fibers of the tubular fiber reinforcing sleeve may be made from a variety of materials. For example, the fibers may be made of a high-strength material. For example, they can be made of metals such as stainless steel and Nitinol, or polymers such as an aramid available under the trade designation KEVLAR, an aromatic polyester liquid crystal polymer available under the trade designation VECTRAN, ultra-high-molecular-weight polyethylene available under the trade designations SPECTRA and DYNEEMA, polyethylene terephthalate (PET) available under the trade designation DACRON, poly(p-phenylene-2,6-benzobisthiazole) available under the trade designation TERLON (PBT), poly(p-phenylene-2,6-benzobisoxazole) available under the trade designation ZYLON (PBO), a polyimide, a polyamide, or other polyethylenes, aramids, polyesters, and the like. If desired, mixtures of fibers may be used to achieve the desired properties in the overall braid or other reinforcement configuration of the tubular fiber reinforcing sleeve.

In certain embodiments, the fibers of the tubular fiber reinforcing sleeve may be made from a material with shape memory properties so that the balloon incorporating the material can be caused to change from one configuration to another upon subjecting the balloon to a temperature change. In this way, the change of configuration of the fiber reinforcement (e.g., braid) can be used to assist the deployment or contraction of the balloon. Typical materials which possess such a memory function are medical grade Nitinol and polymers such as polyesters, notably PET, polyamides, polyurethanes, or polynorbornene.

The fibers of the tubular fiber reinforcing sleeve can be over a broad range of thicknesses. For example, fiber thicknesses can be at least 0.012 mm. For example, fiber thicknesses can be no greater than 0.12 mm.

Balloons of the present disclosure may be compliant, non-compliant, or semi-compliant. Preferably, the balloons are non-compliant or semi-compliant.

This classification is based upon the operating characteristics of the individual balloon, which in turn depend upon the process used in forming the balloon, as well as the material used in the balloon forming process. All types of balloons provide advantageous qualities. A balloon which is classified as “non-compliant” is characterized by the balloon's inability to grow or expand appreciably beyond its rated or nominal diameter. Non-compliant balloons are referred to as having minimal distensibility. In balloons currently known in the art (e.g., polyethylene terephthalate), this minimal distensibility results from the strength and rigidity of the molecular chains which make up the base polymer, as well as the orientation and structure of those chains resulting from the balloon formation process.

A balloon which is referred to as being “compliant” is characterized by the balloon's ability to grow or expand beyond its nominal or rated diameter. In balloons currently known in the art (e.g., polyethylene, polyvinylchloride), the balloon's compliant nature or distensibility results from the chemical structure of the polymeric material used in the formation of the balloon, as well as the balloon forming process. Compliant balloons upon subsequent inflations, will achieve diameters which are greater than the diameters which were originally obtained at any given pressure during the course of the balloon's initial inflation.

A balloon which is referred to as being “semi-compliant” is characterized by low compliance with moderate stretching upon the application of tensile force. Typically, a semi-compliant balloon has a compliance of less than 0.045 millimeters/atmosphere (mm/atm), whereas a compliant balloon has a compliance of greater than 0.045 mm/atm, and a noncompliant balloon has a compliance of not greater than 0.025 mm/atm. Examples of such semi-compliant balloon materials include Nylon 12 and polyethylene block amide co-polymer available under the trade designation PEBAX, including PEBAX 7033.

The term “elastic,” as it is used in connection with this disclosure, refers only to the ability of a material to follow the same stress-strain curve upon the multiple applications of stress. Elasticity, however, is not necessarily a function of how distensible a material is. It is possible to have an elastic, non-distensible material or a non-elastic, distensible material. Preferred balloons of the present disclosure may be used to dilate multiple lesions without compromising primary performance.

Materials used in balloons of the present disclosure are primarily thermoplastics or thermoplastic elastomers. They may be block co-polymers, graft co-polymers, a blend of elastomers and thermoplastics, and the like. Such polymers may be crosslinked or not, but preferably are not crosslinked. Various combinations of polymers may be used in making balloons of the present disclosure.

Exemplary balloon materials used to make the continuous polymer tube of the balloon body can include one or more materials selected from the group consisting of a polyester, a polyamide, a polyethylene, a polyurethane, a polycaprolactone, a polyimide, a polyether, an ionomer, a liquid crystal polymer, and combinations thereof (i.e., blends, mixtures, and copolymers of these with each other and with other monomers/oligomers/polymers). Typically, and preferably, such balloon materials are block copolymers. Examples of mixtures of polymers include mixtures of nylon and polyamide block copolymers and polyethylene terephthalate and polyester block copolymers.

In certain embodiments, the polymers may include polyethylene terephthalate polymers and polybutylene terephthalate polymers. Other useful materials include polyesterether and polyetheresteramide copolymers such as those described in U.S. Pat. No. 5,290,306 (Trotta et al.), polyether-polyamide copolymers such as those described in U.S. Pat. No. 6,171,278 (Wang et al.), polyurethane block copolymers such as those described in U.S. Pat. Nos. 6,210,364 B1, 6,283,939 B1, and 5,500,180 (all to Anderson et al.). Suitable polymers also include materials such as the multiblock copolymers of the zero-fold balloon described in U.S. Pat. Pub. No. 2005/0118370.

Particularly preferred non-compliant and semi-compliant balloons include a polyethylene terephthalate, a polybutylene terephthalate, a polyamide, a polyether block amide, a polyblend comprising a polyamide, a polyblend comprising a polyethylene terephthalate, a polyblend comprising a polybutylene terephthalate, a multi-layer construction comprising a polyamide layer, a multi-layer construction comprising a polyethylene terephthalate layer, or a multi-layer construction comprising a polybutylene terephthalate layer.

In certain embodiments, because the tubular fiber reinforcing sleeve will provide mechanical support and strength to the balloon body, the material of the balloon body can be one which would not on its own survive the expansion conditions. Thus, it is possible to use a softer and physiologically more acceptable polymer than, for example, PET, such as, for example a vinylic or polyalkylene polymer, or a polyurethane.

If desired the wall of the balloon body may be of a composite or laminated construction with an outer layer of a soft polymer, for example, a medical grade polyurethane; and an inner layer of a fluid resistant polymer, for example a PET or polyvinylidene chloride.

A reinforced balloon of the invention can be of any suitable size and shape having regard to the use to be made of the balloon. It will usually be preferred that the balloon adopt a cylindrical configuration when deployed. It can be used upon a tubular catheter which has an external diameter of at least 0.5 millimeters (mm), and often up to 1.5 mm or more. The external diameter of a deployed but unstretched balloon body is typically at least 1.5 times that of the tubular catheter, for example, from 1 to 10 millimeters. Similarly, the balloon body can be of any suitable axial length including, for example, 300 mm or more. An exemplary balloon can have an outer diameter of 6 mm, an inner diameter of 5.9 mm, and a length of 25 mm, in an as delivered or wrapped state. These dimensions may be larger or smaller, depending on the application for the balloon catheter. Dimensions of a reinforced balloon are the same as above.

In certain embodiments, the reinforcement is applied to a balloon of the present disclosure during the preparation of the balloon, as opposed to an already formed balloon (e.g., as described in U.S. Pat. Pub. No. 2008/0183132 (Davies et al.)). For example, a tubular fiber reinforcing sleeve (e.g., a polyester braided sleeve 3 mm in diameter) is longitudinally stretched to reduce its outer diameter (OD). Stretching longitudinally reduces the sleeve OD thus facilitating placement of the balloon tube/sleeve combination into the balloon mold (which typically has to be done by loading the tube/sleeve combination into the mold through the narrow balloon neck regions). The amount of longitudinal stretching is typically 2× to 5× the length (e.g., from 20 mm to 70 mm). Generally, very little force is used to stretch the sleeve because all that is happening is that the helix angle is being changed from, for example, 50-60 degrees (unstretched), to a helix angle of, for example, 10-20 degrees (fully stretched). This amount of stretching can easily be performed by hand.

FIG. 2 shows a general process of making a reinforced dilatation balloon that includes the steps of: providing a tubular balloon parison and a tubular fiber reinforcing sleeve; axially stretching the tubular fiber reinforcing sleeve; applying the tubular, axially stretched, fiber reinforcing sleeve to the tubular balloon parison; applying a matrix polymer to the tubular fiber reinforcing sleeve, the tubular balloon parison, or both, to form a balloon parison/sleeve/matrix polymer construction; placing the balloon parison/sleeve/matrix polymer construction in a balloon-forming mold; and expanding the balloon parison/sleeve/matrix polymer construction and applying heat to form a fiber reinforced balloon.

In certain embodiments, a low melting matrix polymer is applied to the tubular fiber reinforcing sleeve in its longitudinally stretched state, which is then applied to a balloon parison. Alternatively, a low melting matrix polymer can be applied to the combination of the sleeve/balloon parison. Other methods of forming a balloon parison/sleeve/matrix polymer construction can be used as would be clear to one of skill in the art upon reading the present disclosure.

This can be done by applying such polymer, or mixture of components (e.g., monomers or oligomers) to form a polymer, to the tubular fiber reinforcing sleeve, to the balloon parison, or both, (which can be accomplished, e.g., before or after combining the sleeve and parison, before or after axially stretching the sleeve), using a variety of methods. This can be done by dip coating, spray coating, or using a wide variety of other coating techniques. For example, a low melting matrix polymer can be applied from a solution or dispersion of a polymer in an appropriate solvent (e.g., THF) over a wide range of concentrations (e.g., from a very dilute solution up to a concentration of 25% by weight). For example, a mixture of 6 wt-% polyurethane in THF (tetrahydrafuran) can be used. This is then dried to allow the solvent to flash off over a period of a couple of hours, for example. It is possible to use a system that involves polymerizing and/or crosslinking a polymer in situ once coated, optionally in a solvent for viscosity control, for example. If such a system is used, the polymerization and/or crosslinking could be promoted using a light curing process via placement of the coated device into a UV light curing oven.

If a matrix polymer is applied to the tubular fiber reinforcing sleeve, it can be done before, although it is typically done after, the tubular fiber reinforcing sleeve is longitudinally stretched. Alternatively, the tubular reinforcing sleeve in its longitudinally stretched state can be applied to a balloon parison previously coated with a relatively low melting matrix polymer. Alternatively, and preferably, both the tubular fiber reinforcing sleeve and the balloon parison can be coated with a relatively low melting matrix polymer.

However the three are combined (matrix polymer, balloon parison, and tubular reinforcing sleeve, the balloon parison/sleeve/matrix polymer construction is inserted into a balloon mold and subjected to a stretch blow molding process. Such processes are well known in the balloon manufacturing process to impart orientation into the balloon material; however, prior to the present disclosure fiber reinforcing sleeves have never been applied to a balloon during this balloon forming process.

Any of a wide variety of balloon molds can be used. For example, a standard mold can be used, typically one that requires the tube/braid construct to be loaded from one balloon end through the mold neck and out the other mold neck. These molds either are one piece designs (they do not split at all) or they split radially at one or more points. There are other mold types known in the industry that are of a clamshell design where the mold splits along its entire length to form two symmetrical halves. If using these types of molds, the tube/braid construct could be placed into the mold (with the mold split open) without needing to feed the material through the balloon neck.

The balloon parison/sleeve/matrix polymer construction is exposed to a heating step during the balloon forming process to cause fusing (i.e., welding) of the polymer coating/sleeve onto the underlying balloon parison. This temperature is typically no greater than 125° C., and often no greater than 150° C. Such high temperatures are possible because the construction is constrained from dimensional movement (either by shrinkage or expansion) while the high temperature acts to at least partially melt the low melting matrix polymer and allow it to fuse itself to the fiber reinforcing sleeve and the underlying balloon material. However, use of very high temperatures greater than 150° C. to weld or fuse the matrix polymer to the base balloon risks annealing (thus weakening) the base balloon by allowing relaxation of the orientation effects that give the balloon its strength. The resultant thermally bonded balloon/sleeve composite is then removed from the mold.

Suitable “low melting” matrix polymers are those that melt at a temperature, or somewhat below a temperature, used in a typical balloon blow molding manufacturing process. An advantage of the process of the invention is that the heat and pressure combination within a balloon mold can be used to achieve thermal bonding of the matrix/fiber construction to the balloon, where such high pressures, and especially high temperatures, could not be used on an already formed balloon. This expands the range of potential matrix materials.

A matrix polymer can be selected from a wide range of flexible polymers that can be applied in thin layers (by dipping, spraying, etc.), that have a melting temperature well above ambient temperature or temperatures that the matrix is likely to encounter in manufacturing, storage, or use (e.g., greater than 75° C.). Preferably, the melting temperature of such material is less than 150° C., and more preferably, less than 125° C.

Suitable such materials include, for example, polyurethanes including an aliphatic polyurethane, such as an aliphatic polyether-based polyurethane, or an aromatic polyurethane, for example, polyurethanes available from CT Biomaterials as CHRONOFLEX AL, CHRONOFLEX AR and CHRONOFLEX C, and available from Thermedics Polymer Products as TECOFLEX TPU, TECOTHANE TPU, CARBOTHANE TPU, TECOPHILIC TPU, TECOPLAST TPU, TECOFLEX SG-80A, TECOFLEX SP-80A-150, TECOFLEX SG-85A, TECOFLEX SP-93A-100, TECOFLEX SG-93A, TECOFLEX SP-60D-60 and TECOFLEX SG-60D. Others include polyvinylidene fluorides, polyolefins, polyamides, polyesteretheramide copolymers available as PEBAX polymers. Various combinations of matrix polymers can be used if desired.

The selection of the matrix polymer, the fibers of the fiber reinforcing sleeve, and the balloon body are selected such that the matrix polymer at least partially melts during the blow molding balloon manufacturing process, but the fibers and balloon body material do not. It is desirable to select a matrix polymer that does not have a melting temperature close to that of the fibers or balloon body to avoid the latter from losing the orientation effects that provide the desired tensile or burst strength. Thus, in this context, “low melting” means that the matrix polymer has a lower melting point than the fibers of the tubular fiber reinforcing sleeve and a lower melting point than the material of the balloon body. Preferably, the matrix polymer melting point is at least 25° C. lower than that of the fibers of the tubular fiber reinforcing sleeve and the material of the balloon body.

Suitable solvents include THF (tetrahydrafurn), xylene, acetone, water, alcohols, etc. The choice of solvent can be readily determined by one of skill in the art depending on the specific polymer selected as the matrix polymer.

The process described herein provides a better fit between the tubular fiber reinforcing sleeve and the balloon body than can typically be accomplished when applying the same tubular fiber reinforcing sleeve to a preformed balloon. For example, it is difficult to form a snug fit between the sleeve and cone regions (e.g., regions 10 and 11 in FIG. 1). Because the balloon is blown up inside the tubular fiber reinforcing sleeve, the sleeve is forced to conform to the balloon shape, and then is held securely in place by the application of heat and a fusing process before the composite exits the mold. Also, because the process described herein uses a blow molding process that uses relatively high temperatures and pressures, the balloon profile can be better controlled and reduced. That is, the application of such temperatures and pressures used during a blow molding process allow for the polymeric parison/sleeve/matrix polymer constructions to be molded into a “sandwich” or laminate composite having a relatively small thickness. For example, an exemplary balloon body wall thickness formed in a blow molding process is 0.012 mm to 0.025 mm, and an exemplary reinforced balloon body wall thickness formed in a blow molding process according to the present disclosure is 0.04 mm to 0.1 mm.

In accordance with an embodiment of this disclosure, the balloons are formed from a thin wall parison of a polymeric material using a mold which can be provided with a heating element. In a preferred embodiment, the mold receives a tubular parison made of a polymeric material having one or more tubular fiber reinforcing sleeves pre-bonded thereto using a matrix polymer. The ends of the parison/sleeve/matrix polymer construction extend outwardly from the mold and one of the ends is sealed while the other end is affixed to a source of inflation fluid, typically nitrogen gas, under pressure. Clamps or “grippers” are attached to both ends of the parison/sleeve/matrix construction so that the parison/sleeve/matrix polymer construction can be drawn apart axially in order to axially stretch the parison/sleeve/matrix polymer construction while at the same time said parison/sleeve/matrix polymer construction is capable of being expanded radially or “blown” with the inflation fluid. The radial expansion and axial stretch step or steps may be conducted simultaneously, or depending upon the polymeric materials of which the parison/sleeve/matrix polymer construction is made, following whatever sequence is required to form a reinforced balloon.

Typically, failure to axially stretch the parison during the balloon forming process will result in a balloon that will have an uneven wall thickness and will exhibit a wall tensile strength lower than the tensile strength obtained when the parison is both radially expanded and axially stretched.

The radial expansion and axial stretching can also improve the overall strength of the reinforced balloon by inducing additional orientation in the tubular fiber reinforcing sleeve (in the fibers themselves). For example, if making a reinforced Nylon balloon, then a tubular fiber reinforcing sleeve made of a similar Nylon could be designed in an “undersized” manner such that the fibers of the sleeve are then forced to stretch in the forming process (under the influence of the heat/pressure combination used to form the balloon), thus gaining strength in the desired (generally radial) direction. This also can ensure sizing of the fiber reinforcement (e.g., braid) to the desired balloon outer diameter.

The polymeric parison/sleeve/matrix polymer constructions used in this disclosure are preferably drawn axially and expanded radially simultaneously within the mold. To improve the overall properties of the balloons formed, it is desirable that the parison/sleeve construction is axially stretched and blown at temperatures above the glass transition temperature (Tg) of the polymeric material used to form the parison. If it is intended/desired to impart extra orientation to the fibers in the molding process, then the temperature of the fibers also must be above Tg. If it is not intended to impart extra orientation, but only to fully expand the fiber sleeve into contact with the mold walls, then the fibers can remain at a temperature below their Tg. This expansion usually takes place at a temperature of 80° C. to 150° C., depending upon the materials used in the process.

In accordance with this disclosure, based upon the polymeric material used in the parison, the parison is dimensioned with respect to the intended final configuration of the balloon. It is particularly important that the parison have relatively thin walls. The wall thickness is considered relative to the inside diameter of the parison which has wall thickness-to-inside diameter ratios of less than 0.6, and preferably between 0.57 and 0.09 or even lower. The use of a parison with such thin walls enables the parison to be stretched radially to a greater and more uniform degree because there is less stress gradient through the wall from the surface of the inside diameter to the surface of the outside diameter. By utilizing a parison which has thin walls, there is less difference in the degree to which the inner and outer surfaces of the tubular parison are stretched.

Preferably, the parison is drawn from a starting length L1 to a drawn length L2, which preferably is between about 1.10 to about 6 times the initial length L1. The tubular parison, which has an initial internal diameter ID1 and an outer diameter OD1, is expanded by the inflation fluid emitted under pressure to the parison to an internal diameter ID2, which is preferably 5 to 8 times the initial internal diameter ID1, and an outer diameter OD2, which is about equal to or preferably greater than about 3 times the initial outer diameter OD1. The parison is preferably subjected to between 1 and 5 cycles during which the parison is axially stretched and radially expanded with an elevated inflation pressure (i.e., a pressure sufficient to inflate the balloon), preferably an elevated pressure of at least 100 psi, and preferably up to 1000 psi. Nitrogen gas is the preferable inflation fluid for the radial expansion step.

Following the initial expansion step, the expanded parison/sleeve construction is subjected to a “Heat Set” step, preferably while maintaining the elevated inflation pressure of at least 100 psi and more preferably up to 1000 psi. The temperature chosen for the “Heat Set” step is one that induces crystallization and “freezes” or “locks” the orientation of the polymer chains which resulted from axially stretching and radially expanding the parison/sleeve construction. The temperatures which can be used in this heat set step are therefore dependent upon the particular polymeric material used to form the parison/sleeve construction and the ultimate properties desired in the balloon product (e.g., distensibility, strength, and compliancy), as well as the material of the matrix polymer (as discussed above). The temperatures chosen for this “Heat Set” step will more usually be above the temperature used during the initial expansion step but will be below the melting temperature of the melt temperature of the polymeric materials from which the parison and fiber reinforcing sleeves are formed. The heat set step ensures that the expanded parison/fiber reinforcing sleeve and the resulting reinforced balloon will have temperature and dimensional stability.

The reinforced balloon thus formed may be removed from the mold, sterilized, and affixed to a catheter. In certain embodiments, following balloon formation, and prior to mounting on the catheter, one taper/cone region of the balloon can be trimmed completely off the balloon (distal balloon region) while the other taper/cone region remains to form one of the bond regions. The other bond region of the balloon can be part of the balloon body.

### ILLUSTRATIVE EXAMPLES

1. A reinforced dilatation balloon comprising:

a balloon body having a continuous polymer tube having a proximal end, a distal end, and an external surface; and

a tubular fiber reinforcing sleeve thermally bonded along the length of the sleeve to at least a portion of the external surface of the balloon body.

2. The reinforced dilatation balloon of embodiment 1 wherein the external surface of the continuous tube of the balloon body further comprises at least one matrix polymer disposed thereon.

3. The reinforced dilatation balloon of embodiment 2 wherein the matrix polymer is selected from the group consisting of a polyurethane, a polyvinylidene fluoride, a polyolefin, a polyamide, a polyesteretheramide copolymer, and combinations thereof.

4. The reinforced dilatation balloon of any one of embodiments 1 through 3 wherein the continuous polymer tube of the balloon body comprises one or more materials selected from the group consisting of a polyethylene terephthalate, a polybutylene terephthalate, a polyamide, a polyether block amide (which can be in the form of a blend or a multi-layer construction, such as a polyblend comprising a polyamide, a polyblend comprising a polyethylene terephthalate, a polyblend comprising a polybutylene terephthalate, a multi-layer construction comprising a polyamide layer, a multi-layer construction comprising a polyethylene terephthalate layer, or a multi-layer construction comprising a polybutylene terephthalate layer).

5. The reinforced dilatation balloon of any one of embodiments 1 through 4 wherein the tubular fiber reinforcing sleeve comprises one or more fiber reinforcing sleeves.

6. The reinforced dilatation balloon of embodiment 5 wherein the tubular fiber reinforcing sleeve comprises a plurality of fiber reinforcing sleeves disposed on the balloon body.

7. The reinforced dilatation balloon of embodiment 5 wherein the tubular fiber reinforcing sleeve comprises one fiber reinforcing sleeve disposed on the balloon body and extending along the entire length of the balloon body.

8. The reinforced dilatation balloon of any one of embodiments 1 through 7 wherein the tubular fiber reinforcing sleeve comprises braided fibers.

9. The reinforced dilatation balloon of embodiment 8 wherein the braided fibers comprise fibers made of a material selected from the group consisting of stainless steel, Nitinol, an aramid polymer, an aromatic polyester liquid crystal polymer, an ultra-high-molecular-weight polyethylene, a polyethylene terephthalate, a poly(p-phenylene-2,6-benzobisthiazole), a poly(p-phenylene-2,6-benzobisoxazole), a polyimide, a polyamide, a polyethylene, polyester, and mixtures thereof.

10. The reinforced dilatation balloon of embodiment 1 wherein the balloon body comprises a non-compliant or semi-compliant balloon body.

11. A method of making a reinforced dilatation balloon, the method comprising:

providing a tubular balloon parison comprising a polymeric material;

providing a tubular fiber reinforcing sleeve;

axially stretching the tubular fiber reinforcing sleeve;

applying the tubular, axially stretched, fiber reinforcing sleeve to the tubular balloon parison;

applying a matrix polymer to the tubular fiber reinforcing sleeve, the tubular balloon parison, or both, to form a balloon parison/sleeve/matrix polymer construction;

providing a mold for forming a balloon;

placing the balloon parison/sleeve/matrix polymer construction in the mold; and

expanding the balloon parison/sleeve/matrix polymer construction and applying heat to form a fiber reinforced balloon.

12. The method of embodiment 11 wherein: expanding the balloon parison/sleeve/matrix polymer construction comprises axially stretching and radially expanding the balloon parison/sleeve/matrix polymer construction at a temperature above the Tg of the polymeric material and at an elevated inflation pressure.

13. The method of embodiment 11 or embodiment 12 wherein applying a matrix polymer to the tubular fiber reinforcing sleeve, the tubular balloon parison, or both, to form a balloon parison/sleeve/matrix polymer construction comprises applying a matrix polymer to the tubular fiber reinforcing sleeve before applying the sleeve to the parison.

14. The method of any one of embodiments 11 through 13 wherein applying a matrix polymer to the tubular fiber reinforcing sleeve, the tubular balloon parison, or both, to form a balloon parison/sleeve/matrix polymer construction comprises applying a matrix polymer to the tubular fiber reinforcing sleeve after axially stretching the tubular fiber reinforcing sleeve.

15. The method of any one of embodiments 11 through 14 wherein applying a matrix polymer comprises coating a matrix polymer out of a solution or dispersion of the matrix polymer in a solvent.

### EXAMPLES

Objects and advantages of this disclosure are further illustrated by the following examples, but the particular materials and amounts thereof recited in these examples, as well as other conditions and details, should not be construed to unduly limit this disclosure.

### Example 1

A polyester braided sleeve (3 mm diameter) available from Secant Medical Polyester, Perkasie, Pa., was manually longitudinally stretched to about 3.5 times its original length to minimize the outer diameter. The braided longitudinally stretched sleeve was coated with a solution of a polyurethane available under the trade designation TECOTHANE SG 80A (Thermedics, Polymer Products, Wilmington, Mass.) in THF (6% by weight of the polymer in the solvent), and dried at room temperature and humidity for a period sufficient to ensure evaporation of all solvent (approximately 6 hours) while in the longitudinally stretched configuration. The braided longitudinally stretched sleeve was placed over a plasticised Nylon-12 balloon parison (Hypertension 4.5×3 mm AV2000 design). The balloon parison/braided sleeve construction was inserted into a balloon mold (a standard mold that requires the tube/braid construct to be loaded from one balloon end through the mold neck and out the other mold neck). The balloon/braided sleeve was exposed to a heat step (150° C. for 45 seconds) to cause fusing of the polymer coating/braided sleeve onto the underlying balloon.

This balloon/braided sleeve was formed into a balloon inside the balloon mold by application of heat (150° C.), internal pressure (400 psi), with longitudinal stretching by methods well known in the industry. Following this initial balloon forming step, the balloon was subjected to a heat setting step, while the internal pressure was maintained. For some combinations of balloon/braid/polymer coating materials this heat set step acts to both heat set the balloon and to thermally fuse the coating/braid to the underlying balloon. For other balloon/braid/polymer coating combinations a separate additional step may be needed in the balloon forming process, utilizing an alternative (generally higher) temperature in order to soften the polymer coating sufficiently to fuse it to the base balloon.

The thermally bonded balloon/braided sleeve composite was removed from the mold. The composite showed excellent adhesion of the braid to the underlying balloon.

### Example 2

A braided sleeve made of helically wound Nylon 6 fibers with helix angle of 54 degrees (sleeve OD in an unstretched state is 4 mm) is longitudinally stretched by about 3.5 times to reduce its OD to about 1.5 mm with helix angle of about 15 degrees. This stretched sleeve is loaded over a balloon preform made of PEBAX 7033 with ID of 0.024 inch and OD 0.046 inch. This combination is then dip coated in a solution of 3 wt-% polyurethane in THF. This material is then allowed to dry by evaporation for about 6 hours or more to drive off the solvent. The preform/braid/polymer coating is then inserted into a 4 mm ID balloon mold and subjected to a combination of heat (120° C.), internal pressure (350 psi) and longitudinal stretching (approximately 2×) to form a balloon within the mold. Finally, a heat set step is applied to the balloon within the mold which acts both to heat set the balloon and to thermally fuse the polymer coating to the base balloon material. This step is performed at 140° C. with internal pressure of 450 psi for about 45 seconds. Following mold cooling, the balloon/braid composite balloon is removed from the balloon mold.

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

What is claimed is:

1. A reinforced dilatation balloon comprising:
a balloon body having a continuous polymer tube having a proximal end, a distal end, and an external surface; and
a tubular fiber reinforcing sleeve thermally bonded along the length of the sleeve to at least a portion of the external surface of the balloon body.

2. The reinforced dilatation balloon of claim 1 wherein the external surface of the continuous tube of the balloon body further comprises at least one matrix polymer disposed thereon.

3. The reinforced dilatation balloon of claim 2 wherein the matrix polymer is selected from the group consisting of a polyurethane, a polyvinylidene fluoride, a polyolefin, a polyamide, a polyesteretheramide copolymer, and combinations thereof.

4. The reinforced dilatation balloon of claim 1 wherein the continuous polymer tube of the balloon body comprises one or more materials selected from the group consisting of a polyethylene terephthalate, a polybutylene terephthalate, a polyamide, a polyether block amide.

5. The reinforced dilatation balloon of claim 1 wherein the tubular fiber reinforcing sleeve comprises one or more fiber reinforcing sleeves.

6. The reinforced dilatation balloon of claim 5 wherein the tubular fiber reinforcing sleeve comprises a plurality of fiber reinforcing sleeves disposed on the balloon body.

7. The reinforced dilatation balloon of claim 5 wherein the tubular fiber reinforcing sleeve comprises one fiber reinforcing sleeve disposed on the balloon body and extending along the entire length of the balloon body.

8. The reinforced dilatation balloon of claim 1 wherein the tubular fiber reinforcing sleeve comprises braided fibers.

9. The reinforced dilatation balloon of claim 8 wherein the braided fibers comprise fibers made of a material selected from the group consisting of stainless steel, Nitinol, an aramid polymer, an aromatic polyester liquid crystal polymer, an ultra-high-molecular-weight polyethylene, a polyethylene terephthalate, a poly(p-phenylene-2,6-benzobisthiazole), a poly(p-phenylene-2,6-benzobisoxazole), a polyimide, a polyamide, a polyethylene, polyester, and mixtures thereof.

10. The reinforced dilatation balloon of claim 1 wherein the balloon body comprises a non-compliant or semi-compliant balloon body.

11. A method of making a reinforced dilatation balloon, the method comprising:
providing a tubular balloon parison comprising a polymeric material;
providing a tubular fiber reinforcing sleeve;
axially stretching the tubular fiber reinforcing sleeve;
applying the tubular, axially stretched, fiber reinforcing sleeve to the tubular balloon parison;
applying a matrix polymer to the tubular fiber reinforcing sleeve, the tubular balloon parison, or both, to form a balloon parison/sleeve/matrix polymer construction;
providing a mold for forming a balloon;
placing the balloon parison/sleeve/matrix polymer construction in the mold; and
expanding the balloon parison/sleeve/matrix polymer construction and applying heat to form a fiber reinforced balloon.

12. The method of claim 11 wherein:
expanding the balloon parison/sleeve/matrix polymer construction comprises axially stretching and radially expanding the balloon parison/sleeve/matrix polymer construction at a temperature above the Tg of the polymeric material and at an elevated inflation pressure.

13. The method of claim 11 wherein:
applying a matrix polymer to the tubular fiber reinforcing sleeve, the tubular balloon parison, or both, to form a balloon parison/sleeve/matrix polymer construction comprises applying a matrix polymer to the tubular fiber reinforcing sleeve before applying the sleeve to the parison.

14. The method of claim 13 wherein:
applying a matrix polymer to the tubular fiber reinforcing sleeve, the tubular balloon parison, or both, to form a balloon parison/sleeve/matrix polymer construction comprises applying a matrix polymer to the tubular fiber reinforcing sleeve after axially stretching the tubular fiber reinforcing sleeve.

15. The method of claim 11 wherein applying a matrix polymer comprises coating a matrix polymer out of a solution or dispersion of the matrix polymer in a solvent.
